(19) United States
(12) Patent Application Publication
(10) Pub. No.: US 2003/0198664 A1
(43) Pub. Date: Oct. 23, 2003

(54) Lipid mediated screening of drug candidates for identification of active compounds

(76) Inventors: Sean Michael Sullivan, Gainesville, FL (US); Robert A. Copeland, Hockessin, DE (US)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 10/404,896

(22) Filed: Mar. 31, 2003

Related U.S. Application Data
(60) Provisional application No. 60/368,529, filed on Mar. 29, 2002.

(51) Int. Cl. C12Q 1/68; G01N 33/574; A61K 9/127; C12N 15/88

(52) U.S. Cl. 424/450; 435/6; 435/7.23; 435/458

(57) ABSTRACT

The subject invention provides liposome formulations that are capable of specifically targeting cell types. The subject invention also provides for the encapsulation of new chemical entities (NCE) or other drug candidate molecules (DCM) within liposomes that specifically target a particular cell type. The subject invention, advantageously, solubilizes compounds, with low solubility in aqueous environments, and permits screening of these compounds against intact cells for biological activity in the absence of detergents that can damage cell membranes. Also provided are methods of preparing liposome formulations that target a specific cell type and methods of delivering therapeutic agents to target cells.

### CROSS-REFERENCE TO RELATED APPLICATION

The application claims priority to U.S. Provisional Application Serial No. 60/368,529, filed Mar. 29, 2002, which is hereby incorporated by reference in its entirety (including all figures, amino acid and polynucleotide sequences, and formulae).

### BACKGROUND OF THE INVENTION

Liposomes are microscopic vesicles having single or multiple phospholipid bilayers that can be used to entrap compounds. Liposomes with multiple bilayers are known as multilamellar vesicles (MLVs); liposomes with a single bilayer are known as unilamellar vesicles (UV). Liposomes have been formed in sizes as small as tens of Angstroms to as large as a few microns. Most liposomes are non-toxic, non-antigenic and biodegradable in character since they have the molecular characteristics similar to mammalian membranes.

The advent of combinatorial chemistry, combinatorial libraries, compound libraries, and automated synthesis methods has significantly expanded the numbers of potentially beneficial therapeutic compounds produced in the pharmaceutical industry. After these compounds have been synthesized, they are, typically, screened for their ability to impact a particular process, target molecule, or molecular interaction. Compounds successfully demonstrating a desired effect are then tested for biological activity in cell based screening systems.

Many compounds exhibiting desirable characteristics are lost to drug development because of failure in the cell-based screening assays. Failure can, often, be attributed to a lack of solubility in aqueous environments or lack of cellular uptake. Failed compounds are often discarded in the drug discovery process because the time and expense required to increase drug solubility via medicinal chemistry or because the time and expense of identifying properties that reduce or abrogate cellular uptake is prohibitive.

Other compounds are discarded in drug development programs because of a failure to meet acceptance criteria for new chemical entity (NCE) development. Acceptance criteria that eliminate an NCE include undesirable physiochemical properties (e.g., poor activity in a variety of delivery means), poor water solubility, manufacturing issues, regulatory issues, and/or marketing issues.

The subject invention addresses issues in the drug discovery process and provides liposome formulations and methods that facilitate the screening of compounds in cell-based screening systems. The invention has applicability to virtually all compounds and can be used to specifically target a variety of cell types.

### BRIEF SUMMARY

The subject invention provides liposome formulations that are capable of specifically targeting cell types. The subject invention also provides for the encapsulation of new chemical entities (NCE) or other drug candidate molecules (DCM) within liposomes that specifically target a particular cell type. The subject invention, advantageously, solubilizes compounds, with low solubility in aqueous environments, and permits screening of these compounds against intact cells for biological activity in the absence of detergents that can damage cell membranes. Also provided are methods of preparing liposome formulations that target a specific cell type and methods of delivering therapeutic agents to target cells.

### DETAILED DISCLOSURE

In one embodiment, the subject invention provides a method of preparing liposomes that are cell-type specific (CTSL). One aspect of the invention utilizes targeting agents, such as antibodies, receptors, or ligands for the targeting of the liposome formulations. Other embodiments utilize the chemical composition of the liposome formulations for targeting to specific cell types. In certain aspects of the invention, liposomes contain compounds that are to be screened for biological activity in cell-based assays. Other embodiments provide for uptake of the liposomes via endocytosis; endocytosis can be receptor-mediated or occur in the absence of receptors.

In one embodiment of the invention, liposomes of the invention are prepared from “standard liposomes” (SL) according to methods well known in the art. A “standard liposome” contains phosphatidylcholine (PC) and cholesterol (Chol). In certain preferred embodiments, the “standard liposome” contains dioleoylphosphatidylcholine (DOPC) and cholesterol. Ratios of PC:Chol or DOPC:Chol in “standard liposomes” are, typically, about 2:1 (e.g., about 66.67 mole % PC or DOPC and about 33.33 mole % Chol). To provide liposomes that are taken up by target cells, via endocytosis, additional phospholipid components (AC) are titrated into the “standard liposome” formulation. “Standard liposomes” and/or CTSL can be unilamellar or multilamellar. “Standard liposomes” can also be drug/lipid complexes where the drugs are non-covalently bound to the lipid.

Another aspect of the invention provides for the preparation of CTSL by combining components of the standard liposome formulation, additional phospholipid components, water, ethanol, and marker substances into a suspension. The suspension is dried under vacuum to remove the ethanol and the suspension can be re-hydrated with isotonic solutions and assayed for uptake by a specific cell type. Another embodiment provides for spontaneous liposome formation using lipids with asymmetric acyl chains.

Additional phospholipid components (AC) that are suitable for titration into the “standard liposome” include, but are not limited to, phosphatidylglycerol, phosphatidylethanolamine, phosphatidic acid, phosphatidylinositol, mono-, di-, and triglycerides, gangliosides, sphingomyelin, and cerebrosides. In some embodiments, a single additional component is added into the SL formulation. Other embodiments provide for the addition of more than one additional component into the SL formulation.

Another embodiment allows for the formulation of CTSL encapsulating (containing) agents selected from the group consisting of therapeutic agents, marker substances, NCE, and drug candidates by combining these agents with an ethanol/water/lipid suspension. The lipid component of the suspension is provided in ratios specific for uptake by a specific target cell. Ethanol facilitates freezing of the suspension and can be removed under vacuum. The resultant lipid/agent film can, then, be re-hydrated in isotonic solutions, diluted to varying concentrations, and assayed for biological activity or cell uptake. Optionally, therapeutic agents, NCE, drug candidates, or marker substances can be added to the CTSL at the time the dried CTSL are rehydrated. Drugs containing weak acids or weak bases can be loaded into preformed liposomes using gradients of counterions. Another method of introducing drugs into liposomes is to use preformed liposomes comprising lipids that undergo a phase transition from gel to liquid state. Incubation of the drug with the liposomes at the transition temperature allows the drug to diffuse into the liposomes. Subsequent return to the liquid or gel state temperature traps the drug inside the liposome.

Phospholipids used in the formulation of the liposomes of the invention can contain short chain (C₆ to C₈) or long chain (C₁₀ to C₁₈) fatty acids. Short or long chain phospholipids can also contain saturated or unsaturated fatty acid tails. In various embodiments, the phospholipids contain zero, one, two, or three saturated bonds. Preferred embodiments utilize phospholipids containing zero or one saturated bond. The length of the long chain fatty acids can also vary from 12 to 18 carbons (C₁₂ to C₁₈) in various embodiments. Certain embodiments utilized phospholipids having sixteen to eighteen carbon atoms (C₁₆ to C₁₈) in the fatty acid tail.

Accordingly, the subject invention provides for methods of preparing cell-type specific liposomes (CTSL) comprising the addition of AC into SL and screening the CTSL for uptake by target cells. In this aspect of the invention, AC are added to the SL formulation; however, the amount of PC or DOPC contained in the formulation is decreased in an amount equal to the amount of the added AC. Thus, if about 5 mole % of phosphatidylglycerol is added to an SL formulation, the liposome formulation contains about 61.67 mole % PC or DOPC and about 33.33 mole % Chol. Additional components can be added to SL formulations in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 21, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, and 66 mole % increments. Additional components can also be added to DL in fractional mole percentages as well.

In aspects of the invention where more than one AC is added to an SL formulation, the mole % of PC or DOPC is decreased in amounts equal to the sum of the AC added to the SL formulation. The following tables are provided to illustrate the titration of AC into SL formulations. It should be noted that the following tables are exemplary in nature and do not limit the amounts of AC or the number of AC that can be added to the SL formulations. As would be apparent, varying mole percentages of AC can be used in the formulation of CTSL, and the mole percentages of each AC need not be the same.

TABLE 1
Titration of a single AC into an SL formulation

| Mole % | Mole % | Mole % |
|---|---|---|
| Chol | PC or DOPC | AC1 |
| 33.33 | 61.66 | 5 |
| 33.33 | 56.66 | 10 |
| 33.33 | 51.66 | 15 |
| 33.33 | 46.66 | 20 |
| 33.33 | 41.66 | 25 |

TABLE 2
Titration of multiple AC into an SL formulation

| Mole % | Mole % | Mole % | Mole % |
|---|---|---|---|
| Chol | PC or DOPC | AC1 | AC2 |
| 33.33 | 56.67 | 5 | 5 |
| 33.33 | 46.67 | 10 | 10 |
| 33.33 | 36.67 | 15 | 15 |

TABLE 3
Titration of multiple AC (varying mole %) into an SL formulation

| Mole % | Mole % | Mole % | Mole % |
|---|---|---|---|
| Chol | PC or DOPC | AC1 | AC2 |
| 33.33 | 58.67 | 5 | 3 |
| 33.33 | 51.67 | 8 | 7 |
| 33.33 | 45.67 | 11 | 11 |

CTSL are then screened for uptake by target cells. In this aspect of the invention, target cells are contacted with CTSL containing a marker or other substance that allows for the identification of cells that have endocytosed or internalized the CTSL. Markers suitable for use in this aspect of the invention include fluorescent markers that allow for visualization of CTSL uptake, radioisotopes, dyes, or electron dense materials that can be detected within cells. In another aspect of the invention, a cytotoxic substance can be incorporated into a CTSL of the subject invention. Exemplary cytotoxic substances that can be encapsulated in the liposomes of the invention include, and are not limited to, 5-fluorouracil (5-FU) or 5-fluoroorotate (5-FO). Encapsulation of 5-FU or 5-FO in a liposome that can be endocytosed by the cell results in delivery and killing of the cell due to the incorporation of the nucleoside analog into the cell's DNA, thereby allowing for the identification of those cells that have taken up a CTSL according to the subject invention.

Target cells useful in the practice of the invention include any cell type available from commercial sources, including those cell lines held at The American Type Culture Collection (ATCC), 10801 University Blvd., Manassas, Va. 20110-2209, and disclosed within the catalogs provided by the ATCC. These ATCC catalogs are hereby incorporated by reference in their entireties. Non-limiting examples of such cell lines include cancer cell lines, stem cell lines, and cell lines derived from normal tissues, such as hepatic, gastrointestinal, mucosal, vascular, cardiac, renal, mesenchymal, neural, ocular, bone, dermal, epidermis, muscular, prostate, or pulmonary tissue. In certain other embodiments, target cells can be derived from normal tissues cells (e.g., cultured from normal tissues selected from the group consisting of hepatic, gastrointestinal, mucosal, vascular, cardiac, renal, mesenchymal, neural, ocular, bone, dermal, epidermis, muscular, prostate, and pulmonary tissue). In other embodiments, target cells can be, optionally, engineered, for example by transfection with one, or more, suitable DNA construct(s), to express a specific target macromolecule, or a set of macromolecules, that together constitute a reporter system for measuring a specific intracellular biological activity. Alternatively, target cells can be engineered or transfected with one or more DNA constructs that provide for a metabolic pathway that is to be targeted an NCE or drug candidate that is being tested in the system provided herein.

The subject invention also provides methods of screening active agents selected from the group consisting of NCE; DCM; small molecules; agonists; antagonists; peptides; proteins; and nucleic acids selected from the group consisting of interfering RNA and antisense RNA for biological activity comprising contacting a target cell with a drug candidate or NCE containing CTSL optimized for the target cell. In this aspect of the invention, optimized CTSL containing an active agent are contacted with target cells and observed for biological effects upon the target cells.

Often in early phase drug discovery a hypothesis is formulated regarding the cellular/biological consequences of agonizing or antagonizing a specific intracellular target, such as an enzyme, receptor or other macromolecule. It is common to express the macromolecular target and use this to prepare a cell-free assay system, such as an enzyme activity assay or receptor-ligand binding assay. In this way small molecules, peptides, proteins, nucleic acids and other species can be identified that have the desired agonist or antagonist activity on the target. The subject invention is also applicable to target validation experiments. In this aspect of the invention, it is possible to validate a working hypothesis by applying or contacting a CTSL comprising an agonist, antagonist, NCE, or drug candidate to a relevant cell type (or a cell type engineered to express a desired metabolic pathway, enzyme, receptor, macromolecule, or set of macromolecules) and assay the target cell to determine if the expected cellular/biological consequence occurs. Thus, the subject invention provides methods for overcoming technical issues related to the poor solubility and/or permeability of a NCE, drug candidate, agonist or antagonist under study.

Biological effects within the scope of the invention include agonist or antagonist activity on enzymatic activity, antagonist or agonist activity of ligand/receptor interactions; antagonist or agonist activity for protein/protein or protein/DNA or protein/RNA interaction; or agonist or antagonist activity for interactions of nucleic acids (e.g., DNA/DNA or DNA/RNA interactions). Exemplary ligand/receptor pairs include zinc finger protein/dsDNA (also a protein/DNA interaction), enzyme/substrate, enzyme/cofactor, lectin/carbohydrate, hormone/receptor, or cytokine/receptor.

The subject invention also provides methods of treating an individual comprising the administration of compositions comprising CTSL of the invention to an individual in need of treatment. In this aspect of the invention, the CTSL contain an active agent typically used to treat a diseased tissue or lesion or a therapeutic agent (or combination of therapeutic agents) that has demonstrated superior activity against the diseased tissue or lesion. Thus, CTSL according to the invention are specifically tailored to the targeted cell type of the individual; the targeted cell type is, typically, diseased tissue or tissue isolated from a lesion in that individual. Some embodiments allow for the use of normal tissue from an individual in these treatment methodologies. CTSL specific for the target (or diseased) tissue of lesion are prepared according to the methods described supra. CTSL are then used to encapsulate one or more therapeutic agent and are used to assay the sensitivity of the target (or diseased) tissue or lesion for the therapeutic agent, or combinations of therapeutic agents. The therapeutic agent (or combination of therapeutic agents) exhibiting the highest degree of desired activity are chosen, incorporated into CTSL, and administered to the individual to effect the therapeutic regimen. The methods of the invention can be used to treat a disease, condition, or malignancy. The disease, condition, or malignancy can be selected from the group consisting of neoplasms, blood disorders, immunodeficiencies, the induction of an immune response, fungal infections, bacterial infections, viral infections, vitamin deficiencies, allergies, coagulation disorders, circulatory disorders, angina, protozoal infections, pain management, cardiac disorders, and neuromuscular disorders. Other embodiments provide for the delivery of therapeutic agents that provided sedatives or anesthetics to the individual.

Thus, an individual containing a lesion, such as a tumor or other malignant growth, can be treated by introducing active agent-containing CTSL that have been tailored for the tumor or malignant cells utilizing the screening assays of the invention; the therapeutic regimen of can also be specifically tailored by ex vivo assaying of the target cells, extracted from a lesion, for sensitivity to various therapeutic agents, incorporation of that therapeutic agent, or combination of therapeutic agents, having the highest activity against the target cell into CTSL of the invention, and then administration of these CTSL into an individual. Exemplary active agents suitable for use in the instant invention for the treatment of malignancies include those listed in U.S. Pat. No. 5,770,222, hereby incorporated by reference in its entirety.

In other embodiments, the CTSL of the invention can contain therapeutic agents including, but are not limited to: antineoplastic agents, such as platinum compounds (e.g., spiroplatin, cisplatin, and carboplatin), methotrexate, adriamycin, mitomycin, ansamitocin, bleomycin, cytosine arabinoside, arabinosyl adenine, mercaptopolylysine, vincristine, busulfan, chlorambucil, melphalan (e.g., PAM, L-PAM or phenylalanine mustard), mercaptopurine, mitotane, procarbazine hydrochloride dactinomycin (actinomycin D), daunorubicin hydrochloride, doxorubicin hydrochloride, taxol, mitomycin, plicamycin (mithramycin), aminoglutethimide, estramustine phosphate sodium, flutamide, leuprolide acetate, megestrol acetate, tamoxifen citrate, testolactone, trilostane, amsacrine (m-AMSA), asparaginase (L-asparaginase) Erwina asparaginase, etoposide (VP-16), interferon α-2a, interferon α-2b, teniposide (VM-26), vinblastine sulfate (VLB), vincristine sulfate, bleomycin, bleomycin sulfate, methotrexate, adriamycin, and arabinosyl; blood products such as parenteral iron, hemin, hematoporphyrins and their derivatives; biological response modifiers such as muramyldipeptide, muramyltripeptide, microbial cell wall components, lymphokines (e.g., bacterial endotoxin such as lipopolysaccharide, macrophage activation factor), sub-units of bacteria (such as Mycobacteria, Corynebacteria), the synthetic dipeptide N-acetyl-muramyl-L-alanyl-D-isoglutamine; anti-fungal agents such as ketoconazole, nystatin, griseofulvin, flucytosine (5-fc), miconazole, amphotericin B, ricin, and β-lactam antibiotics (e.g., sulfazecin); hormones such as growth hormone, melanocyte stimulating hormone, estradiol, beclomethasone dipropionate, betamethasone, betamethasone acetate and betamethasone sodium phosphate, vetamethasone disodium phosphate, vetamethasone sodium phosphate, cortisone acetate, dexamethasone, dexamethasone acetate, dexamethasone sodium phosphate, flunisolide, hydrocortisone, hydrocortisone acetate, hydrocortisone cypionate, hydrocortisone sodium phosphate, hydrocortisone sodium succinate, methylprednisolone, methylprednisolone acetate, methylprednisolone sodium succinate, paramethasone acetate, prednisolone, prednisolone acetate, prednisolone sodium phosphate, prednisolone tebutate, prednisone, triamcinolone, triamcinolone acetonide, triamcinolone diacetate, triamcinolone hexacetonide and fludrocortisone acetate; vitamins such as cyanocobalamin neinoic acid, retinoids and derivatives such as retinol palmitate, and α-tocopherol; peptides, such as manganese super oxide dismutase; enzymes such as alkaline phosphatase; anti-allergic agents such as amelexanox; anti-coagulation agents such as phenprocoumon and heparin; circulatory drugs such as propranolol; metabolic potentiators such as glutathione; antituberculars such as para-aminosalicylic acid, isoniazid, capreomycin sulfate cycloserine, ethambutol hydrochloride ethionamide, pyrazinamide, rifampin, and streptomycin sulfate; antivirals such as acyclovir, amantadine azidothymidine (AZT or Zidovudine), ribavirin and vidarabine monohydrate (adenine arabinoside, ara-A); antianginals such as diltiazem, nifedipine, verapamil, erythritol tetranitrate, isosorbide dinitrate, nitroglycerin (glyceryl trinitrate) and pentaerythritol tetranitrate; anticoagulants such as phenprocoumon, heparin; antibiotics such as dapsone, chloramphenicol, neomycin, cefaclor, cefadroxil, cephalexin, cephradine erythromycin, clindamycin, lincomycin, amoxicillin, ampicillin, bacampicillin, carbenicillin, dicloxacillin, cyclacillin, picloxacillin, hetacillin, methicillin, nafcillin, oxacillin, penicillin G, penicillin V, ticarcillin rifampin and tetracycline; antiinflammatories such as diflunisal, ibuprofen, indomethacin, meclofenamate, mefenamic acid, naproxen, oxyphenbutazone, phenylbutazone, piroxicam, sulindac, tolmetin, aspirin and salicylates; antiprotozoans such as chloroquine, hydroxychloroquine, metronidazole, quinine and meglumine antimonate; antirheumatics such as penicillamine; narcotics such as paregoric; opiates such as codeine, heroin, methadone, morphine and opium; cardiac glycosides such as deslanoside, digitoxin, digoxin, digitalin and digitalis; neuromuscular blockers such as atracurium mesylate, gallamine triethiodide, hexafluorenium bromide, metocurine iodide, pancuronium bromide, succinylcholine chloride (suxamethonium chloride), tubocurarine chloride and vecuronium bromide; sedatives (hypnotics) such as amobarbital, amobarbital sodium, aprobarbital, butabarbital sodium, chloral hydrate, ethchlorvynol, ethinamate, flurazepam hydrochloride, glutethimide, methotrimeprazine hydrochloride, methyprylon, midazolam hydrochloride, paraldehyde, pentobarbital, pentobarbital sodium, phenobarbital sodium, secobarbital sodium, talbutal, temazepam and triazolam; local anesthetics such as bupivacaine hydrochloride, chloroprocaine hydrochloride, etidocaine hydrochloride, lidocaine hydrochloride, mepivacaine hydrochloride, procaine hydrochloride and tetracaine hydrochloride; general anesthetics such as droperidol, etomidate, fentanyl citrate with droperidol, ketamine hydrochloride, methohexital sodium and thiopental sodium; and radioactive particles or ions such as strontium, iodide rhenium and yttrium.

In this aspect of the invention, CTSL containing therapeutic agents are administered to an individual via intravenous, intracranial, intraarterial, intralesional, or oral administration routes. It is within the abilities of the skilled medical practioner to select the optimal route of administration, or locale of administration, that would yield the highest therapeutic benefit to the individual being treated.

The term “individual(s)” is defined as a single mammal to which is administered a compound or composition of the present invention. The mammal may be, for example a mouse, rat, pig, horse, rabbit, goat, pig, cow, cat, dog, or human. In a preferred embodiment, the individual is a human.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application. We claim:

1. A method of screening active agents for biological effects on a target cell comprising:
a) contacting a target cell with said cell-type specific liposomes (CTSL) optimized for said target cell comprising an encapsulated active agent selected from the group consisting of NCE; DCM; small molecules; agonists; antagonists; peptides; proteins; and nucleic acids selected from the group consisting of interfering RNA and antisense RNA; and
b) assaying said cells for biological effects associated with said active agents.

2. The method according to claim 1, wherein said cell-type specific liposomes further comprise a marker substance.

3. The method according to claim 2, wherein said marker substance is selected from the group consisting of cytotoxic agents, fluorescent markers, radioisotopes, dyes, and electron dense materials.

4. The method according to claim 3, wherein the cytotoxic agent is 5-fluorouracil or 5-fluoroorotate.

5. The method according to claim 1, wherein said biological effect is agonist or antagonist activity on enzymatic activity; antagonist or agonist activity of ligand/receptor interactions; antagonist or agonist activity for protein/protein or protein/DNA or protein/RNA interaction; or agonist or antagonist activity for interactions of nucleic acids.

6. The method according to claim 5, wherein said biological effect is zinc finger protein/dsDNA interaction, a protein/DNA interaction, enzyme/substrate interaction, enzyme/cofactor interaction, lectin/carbohydrate interaction, hormone/receptor interaction, or cytokine/receptor interaction.

7. The method according to claim 1, wherein said target cell is obtained from: a lesion, a tumors, a malignant growth, cancer cell lines, stem cell lines, hepatic cell lines, gastrointestinal cell lines, mucosal cell lines, vascular cell lines, cardiac cell lines, renal cell lines, mesenchymal cell lines, neural cell lines, ocular cell lines, bone cell lines, dermal cell lines, epidermis cell lines, muscular cell lines, prostate cell lines, pulmonary cell lines, or cells cultured from normal tissues selected from the group consisting of hepatic, gastrointestinal, mucosal, vascular, cardiac, renal, mesenchymal, neural, ocular, bone, dermal, epidermis, muscular, prostate, and pulmonary tissue.

8. The method according to claim 1, wherein said CTSL further comprises targeting agents.

9. The method according to claim 8, wherein said targeting agents are antibodies, receptors, or ligands.

10. The method according to claim 1, wherein said target cell is recombinantly engineered to express an enzyme, enzymatic pathway, metabolite, metabolic pathway, receptor complex, macromolecule, or cell surface ligand.

11. The method according to claim 7, wherein said target cell is recombinantly engineered to express an enzyme, enzymatic pathway, metabolite, metabolic pathway, receptor complex, macromolecule, or cell surface ligand.

12. A method of making cell-type specific liposomes (CTSL) comprising:
a) combining, to form a suspension, water, ethanol, active agents/marker substances, additional components (AC) selected from the group consisting of phosphatidylglycerol, phosphatidylethanolamine, phosphatidic acid, phosphatidylinositol, monoglycerides, diglycerides, triglycerides, gangliosides, sphingomyelin, cerebrosides and combinations thereof to a standard liposome (SL) formulation that comprises: 1) phosphatidylcholine (PC) and cholesterol (Chol) in a PC:Chol ratio of about 2:1; or 2) diolcoylphosphatidylcholine (DOPC) and cholesterol (Chol) in a DOPC:Chol ratio of about 2:1;
b) drying the suspension under vacuum;
c) rehydrating the dried suspension with an isotonic solution; and
d) assaying the rehydrated suspension for uptake of a marker substance by a target cell.

13. The method according to claim 12, wherein said active agent/marker substance is selected from the group consisting of cytotoxic agents, fluorescent markers, radioisotopes, dyes, and electron dense materials.

14. The method according to claim 13, wherein the active agent is 5-fluorouracil or 5-fluoroorotate.

15. The method according to claim 12, wherein said target cell is obtained from: a lesion, a tumor, a malignant growth, cancer cell lines, stem cell lines, hepatic cell lines, gastrointestinal cell lines, mucosal cell lines, vascular cell lines, cardiac cell lines, renal cell lines, mesenchymal cell lines, neural cell lines, ocular cell lines, bone cell lines, dermal cell lines, epidermis cell lines, muscular cell lines, prostate cell lines, pulmonary cell lines, or cells cultured from normal tissues selected from the group consisting of hepatic, gastrointestinal, mucosal, vascular, cardiac, renal, mesenchymal, neural, ocular, bone, dermal, epidermis, muscular, prostate, and pulmonary tissue.

16. The method according to claim 12, wherein said target cell is recombinantly engineered to express an enzyme, enzymatic pathway, metabolite, metabolic pathway, receptor complex, macromolecule, or cell surface ligand.

17. The method according to claim 15, wherein said target cell is recombinantly engineered to express an enzyme, enzymatic pathway, metabolite, metabolic pathway, receptor complex, macromolecule, or cell surface ligand.

18. The method according to claim 12, wherein said CTSL further comprises targeting agents.

19. The method according to claim 18, wherein said targeting agents are antibodies, receptors, or ligands.

20. The method according to claim 12, further comprising the addition of one or more active agents to said CTSL prior to said drying step or during said rehydrating step.

21. The method according to claim 20, wherein said one or more active agents is: an anti-neoplastic agents selected from the group consisting of platinum compounds, 5-fluoroorotate, 5-fluorouracil, methotrexate, adriamycin, mitomycin, ansamitocin, bleomycin, cytosine arabinoside, arabinosyl adenine, mercaptopolylysine, vincristine, busulfan, chlorambucil, melphalan, mercaptopurine, mitotane, procarbazine hydrochloride dactinomycin, daunorubicin hydrochloride, doxorubicin hydrochloride, taxol, mitomycin, plicamycin, arminoglutethimide, estramustine phosphate sodium, flutamide, leuprolide acetate, megestrol acetate, tamoxifen citrate, testolactone, trilostane, amsacrine, asparaginase, etoposide, interferon α-2a, interferon α-2b, teniposide, vinblastine sulfate, vincristine sulfate, bleomycin, bleomycin sulfate, methotrexate, adriamycin, and arabinosyl; blood products selected from the group consisting of parenteral iron, hemin, hematoporphyrins and derivatives thereof; biological response modifiers selected from the group consisting of muramyldipeptide, muramyltripeptide, microbial cell wall components, lymphokines, sub-units of bacteria, and synthetic dipeptide N-acetyl-muramyl-L-alanyl-D-isoglutamine; anti-fungal agents selected from the group consisting of ketoconazole, nystatin, griseofulvin, flucytosine, miconazole, amphotericin B, ricin, and β-lactam antibiotics; substances selected from the group consisting of growth hormone, melanocyte stimulating hormone, estradiol, beclomethasone dipropionate, betamethasone, betamethasone acetate, betamethasone sodium phosphate, vetamethasone disodium phosphate, vetamethasone sodium phosphate, cortisone acetate, dexamethasone, dexamethasone acetate, dexamethasone sodium phosphate, flunisolide, hydrocortisone, hydrocortisione acetate, hydrocortisone cypionate, hydrocortisone sodium phosphate, hydrocortisone sodium succinate, methylprednisolone, methylprednisolone acetate, methylprednisolone sodium succinate, paramethasone acetate, prednisolone, prednisolone acetate, prednisolone sodium phosphate, prednisolone tebutate, prednisone, triamcinolone, triamcinolone acetonide, triamcinolone diacetate, triamicinolone hexacetonide and fludrocortisone acetate; vitamins selected from the group consisting of cyanocobalamin neinoic acid, retinoids and derivatives thereof; manganese super oxide dismutase; alkaline phosphatase; amelexanox; heparin; anti-virals selected from the group consisting of acyclovir, amantadine azidothymidine, ribavirin and vidarabine monohydrate; anti-anginals selected from the group consisting of diltiazem, nifedipine, verapamil, crythritol tetranitrate, isosorbidc dinitrate, nitroglycerin and pentaerythritol tetranitrate; antibiotics selected from the group consisting of dapsone, chloramphenicol, neomycin, cefaclor, cefadroxil, cephalexin, cephradine erythromycin, clindamycin, lincomycin, amoxicillin, ampicillin, bacampicillin, carbenicillin, dicloxacillin, cyclacillin, picloxacillin, hetacillin, methicillin, nafcillin, oxacillin, penicillin G, penicillin V, ticarcillin rifampin and tetracycline; anti-inflammitory agents selected from the group consisting of diflunisal, ibuprofen, indomethacin, meclofenamate, mefenamic acid, naproxen, oxyphenbutazone, phenylbutazone, piroxicam, sulindac, tolmetin, aspirin and salicylates; anti-protozoan agents are selected from the group consisting of chloroquine, hydroxychloroquine, metronidazole, quinine and meglumine antimonate; wherein the antirheumatics are penicillamine; opiates selected from the group consisting of codeine, heroin, methadone, morphine and opium; cardiac glycosides selected from the group consisting of deslanoside, digitoxin, digoxin, digitalin and digitalis; neuromuscular blockers selected from the group consisting of atracurium mesylate, gallamine triethiodide, hexafluorenium bromide, metocurine iodide, pancuronium bromide, succinylcholine chloride, tubocurarine chloride and vecuronium bromide; sedatives selected from the group consisting of amobarbital, amobarbital sodium, aprobarbital, butabarbital sodium, chloral hydrate, ethchlorvynol, ethinamate, flurazepam hydrochloride, glutethimide, methotrimeprazine hydrochloride, methyprylon, midazolam hydrochloride, paraldehyde, pentobarbital, pentobarbital sodium, phenobarbital sodium, secobarbital sodium, talbutal, temazepam and triazolam; local anesthetics selected from the group consisting of bupivacaine hydrochloride, chloroprocaine hydrochloride, etidocaine hydrochloride, lidocaine hydrochloride, mepivacaine hydrochloride, procaine hydrochloride and tetracaine hydrochloride; general anesthetics selected from the group consisting of droperidol, etomidate, fentanyl citrate with droperidol, ketamine hydrochloride, methohexital sodium and thiopental sodium; or radioactive compounds selected from the group consisting of strontium, iodide rhenium and yttrium.

22. A method of treating an individual comprising the administration of a composition comprising a carrier and cell-type specific liposomes (CTSL) containing one or more therapeutic agent to an individual in an amount effective to treat the individual.

23. The method according to claim 22, wherein said method treats a disease, condition, or malignancy.

24. The method according to claim 23, wherein said disease, condition, or malignancy is selected from the group consisting of neoplasms, blood disorders, immunodeficiencies, the induction of an immune response, fungal infections, bacterial infections, viral infections, vitamin deficiencies, allergies, coagulation disorders, circulatory disorders, angina, protozoal infections, pain management, cardiac disorders, and neuromuscular disorders, or wherein the disease, condition, or disorder requires sedation or anesthetics.

25. The method according to claim 22, wherein the therapeutic agents are said one or more therapeutic agents is: an anti-neoplastic agents selected from the group consisting of platinum compounds, 5-fluorouracil, 5-fluoroorotate, methotrexate, adriamycin, mitomycin, ansamitocin, bleomycin, cytosine arabinoside, arabinosyl adenine, mercaptopolylysine, vincristine, busulfan, chlorambucil, melphalan, mercaptopurine, mitotane, procarbazine hydrochloride dactinomycin, daunorubicin hydrochloride, doxorubicin hydrochloride, taxol, mitomycin, plicamycin, aminoglutethimide, estramustine phosphate sodium, flutamide, leuprolide acetate, megestrol acetate, tamoxifen citrate, testolactone, trilostane, amsacrine, asparaginase, etoposide, interferon α2a, interferon α-2b, teniposide, vinblastine sulfate, vincristine sulfate, bleomycin, bleomycin sulfate, methotrexate, adriamycin, and arabinosyl; blood products selected from the group consisting of parenteral iron, hemin, hematoporphyrins and derivatives thereof; biological response modifiers selected from the group consisting of muramyldipeptide, muramyltripeptide, microbial cell wall components, lymphokines, sub-units of bacteria, and synthetic dipeptide N-acetyl-muramyl-L-alanyl-D-isoglutamine; anti-fungal agents selected from the group consisting of ketoconazole, nystatin, griseofulvin, flucytosine, miconazole, amphotericin B, ricin, and β-lactam antibiotics; substances selected from the group consisting of growth hormone, melanocyte stimulating hormone, estradiol, beclomethasone dipropionate, betamethasone, betamethasone acetate, betamethasone sodium phosphate, vetamethasone disodium phosphate, vetamethasone sodium phosphate, cortisone acetate, dexamethasone, dexamethasone acetate, dexamethasone sodium phosphate, flunisolide, hydrocortisone, hydrocortisione acetate, hydrocortisone cypionate, hydrocortisone sodium phosphate, hydrocortisone sodium succinate, methylprednisolone, methylprednisolone acetate, methylprednisolone sodium succinate, paramethasone acetate, prednisolone, prednisolone acetate, prednisolone sodium phosphate, prednisolone tebutate, prednisone, triamcinolone, triamcinolone acetonide, triamcinolone diacetate, triamicinolone hexacetonide and fludrocortisone acetate; vitamins selected from the group consisting of cyanocobalamin neinoic acid, retinoids and derivatives thereof; manganese super oxide dismutase; alkaline phosphatase; amelexanox; heparin; anti-virals selected from the group consisting of acyclovir, amantadine azidothymidine, ribavirin and vidarabine monohydrate; anti-anginals selected from the group consisting of diltiazem, nifedipine, verapamil, crythritol tetranitrate, isosorbidc dinitrate, nitroglycerin and pentaerythritol tetranitrate; antibiotics selected from the group consisting of dapsone, chloramphenicol, neomycin, cefaclor, cefadroxil, cephalexin, cephradine erythromycin, clindamycin, lincomycin, amoxicillin, ampicillin, bacampicillin, carbenicillin, dicloxacillin, cyclacillin, picloxacillin, hetacillin, methicillin, nafeillin, oxacillin, penicillin G, penicillin V, ticarcillin rifampin and tetracycline; anti-inflammitory agents selected from the group consisting of diflunisal, ibuprofen, indomethacin, meclofenamate, mefenamic acid, naproxen, oxyphenbutazone, phenylbutazone, piroxicam, sulindac, tolmetin, aspirin and salicylates; anti-protozoan agents are selected from the group consisting of chloroquine, hydroxychloroquine, metronidazole, quinine and meglumine antimonate; wherein the antirheumatics are penicillamine; opiates selected from the group consisting of codeine, heroin, methadone, morphine and opium; cardiac glycosides selected from the group consisting of deslanoside, digitoxin, digoxin, digitalin and digitalis; neuromuscular blockers selected from the group consisting of attracurium mesylate, gallamine triethiodide, hexafluorenium bromide, metocurine iodide, pancuronium bromide, succinylcholine chloride, tubocurarine chloride and vecuronium bromide; sedatives selected from the group consisting of amobarbital, amobarbital sodium, aprobarbital, butabarbital sodium, chloral hydrate, ethchlorvynol, ethinamate, flurazepam hydrochloride, glutethimide, methotrimeprazine hydrochloride, methyprylon, midazolam hydrochloride, paraldehyde, pentobarbital, pentobarbital sodium, phenobarbital sodium, secobarbital sodium, talbutal, temazepam and triazolam; local anesthetics selected from the group consisting of bupivacaine hydrochloride, chloroprocaine hydrochloride, etidocaine hydrochloride, lidocaine hydrochloride, mepivacaine hydrochloride, procaine hydrochloride and tetracaine hydrochloride; general anesthetics selected from the group consisting of droperidol, etomidate, fentanyl citrate with droperidol, ketamine hydrochloride, methohexital sodium and thiopental sodium; or radioactive compounds selected from the group consisting of strontium, iodide rhenium and yttrium.
